(19) United States
(12) Patent Application Publication
(10) Pub. No.: US 2009/0111091 A1
(43) Pub. Date: Apr. 30, 2009

(54) SPECIMEN PRETREATMENT LIQUID, KIT FOR MEASURING VIRUS, AND METHOD FOR DETECTING VIRUS

(75) Inventors: Kotaro FUJIMOTO, Kobe-shi (JP); Takashi Okamoto, Nagoya-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 12/262,890

(22) Filed: Oct. 31, 2008

(30) Foreign Application Priority Data
Oct. 31, 2007 (JP) 2007-284073

(51) Int. Cl. C12Q 1/70

(52) U.S. Cl. 435/5

(57) ABSTRACT

The invention provides a pretreatment liquid for preparing a sample for measuring a virus included in rhinorrhea or sputum by an immunoassay method using an antibody specifically binding to the virus, comprising a protease inhibitor for inhibiting an influence of a protease to the virus, as well as a virus measurement kit and a virus detection method using the specimen pretreatment liquid.

### FIELD OF THE INVENTION

The present invention relates to a specimen pretreatment liquid as well as a virus measurement kit and a virus detection method using the specimen pretreatment liquid.

### BACKGROUND

Severe acute respiratory syndrome (SARS) is an infectious disease found in recent years. The pathogen causing SARS is confirmed to be a new virus (SARS virus) classified into Coronaviridae. SARS virus is a virus infecting the respiratory organs. The initial symptoms of SARS are influenza-like symptoms such as rapid fever, cough, breath shortness, and respiratory distress. About 1 week after first appearance of fever, the symptom of respiratory distress starts to appear in some cases. At the same time, the ability of the virus to infect other persons is heightened in many cases.

Generally in diagnosis of influenza infection, a specimen such as rhinorrhea or sputum is treated with a pretreatment liquid containing a surfactant, and then an influenza virus-derived antigen (protein) contained in the specimen is measured by an immunoassay method (see, for example, JP-A 2006-189304 and US2004265800).

When influenza virus is measured, the virus can be sufficiently measured by the method described above. However, when SARS virus is measured, the amount of SARS virus contained in a specimen such as rhinorrhea or sputum collected from a SARS patient is very lower than in the case of influenza virus. Accordingly, the accurate measurement of SARS virus by the method described above has been difficult.

### SUMMARY

The scope of the present invention is defined solely by the appended claims, and is not affected to any degree by the statements within this summary.

An object of the present invention is to provide a specimen pretreatment liquid necessary for accurate measurement with improved detection sensitivity in an immunoassay method of measuring a virus such as SARS virus contained in a very small amount in a specimen such as rhinorrhea or sputum.

Another object of the present invention is to provide a kit for measuring a virus and a method for detecting a virus, both of which comprise using the specimen pretreatment liquid described above.

When a virus in a specimen is measured by an immunoassay method, the specimen can be pretreated with the specimen pretreatment liquid of the present invention to improve detection sensitivity. Accordingly, -even a virus such as SARS virus occurring in a very small amount in a specimen can be measured more accurately.

### BRIEF DESCRIPTION OF THE DRAWINGS

FIG. 1 is a graph showing the results in Example 1;

FIG. 2 is a graph showing the results in Example 2; and

FIG. 3 is a graph showing the results in Example 3.

### DETAILED DESCRIPTION OF THE EMBODIMENT

The specimen used in the present embodiment is one which can be collected from the living body and is likely to include SARS virus. Specifically, the specimen comprises rhinorrhea and/or sputum. A method of collecting the specimen is not particularly limited, and a known method can be used. Specifically, an aspirator or a cotton-tipped swab can be used to collect rhinorrhea or sputum.

The specimen pretreatment liquid of the present embodiment contains a protease inhibitor. This protease inhibitor inhibits the influence of a protease to SARS virus in a specimen. Decomposition of SARS virus can thereby be prevented. As the protease inhibitor, it is possible to use a serine protease inhibitor, a cysteine protease inhibitor, an aspartic protease inhibitor, and other protease inhibitors. The serine protease inhibitor includes antithrombin III, TLCK (p-toluenesulfonyl-L-lysinechloromethylketone), TPCK (p-toluenesulfonyl-L-phenylalaninechloromethylketone), heparin cofactor II, aprotinin, trypsin inhibitor, AEBSF (4-(2-aminoethyl) benzenesulfonyl fluoride), PMSF, and the like. The cysteine protease inhibitor includes antipain, E-64, and the like. Both the serine and cysteine protease inhibitors include leupeptin, chymostatin, and the like. The aspartic protease inhibitor includes pepstatin A and the like. Other protease inhibitors include amastatin, bestatin, and the like. Among these protease inhibitors, it is preferable to use a mixture of one or more protease inhibitors selected from the group consisting of a serine protease inhibitor, a cysteine protease inhibitor and an aspartic protease inhibitor. A mixture of two or more protease inhibitors may be used in which separately purchased two or more protease inhibitors have been appropriately mixed. A preparation in which a plurality of commercially available protease inhibitors has been mixed may also be used.

Preferably, the specimen pretreatment liquid further contains a chelating agent. A metal ion is sometimes essential for the activity of the enzyme. The chelating agent can bind to the metal ion to form a chelate compound. Accordingly, the chelating agent, similar to the protease inhibitor, can inhibit the action of the protease. The chelating agent includes sodium or potassium ethylenediaminetetraacetate (EDTA), sodium or potassium 1,3-propanediaminetetraacetate (EDTA), nitrilotriacetic acid, and the like.

Preferably, the specimen pretreatment liquid further contains a surfactant. The surfactant has an action of destroying a viral membrane to expose a viral protein. The surfactant includes anonionic surfactant, an anionic surfactant, a cationic surfactant, and an amphoteric surfactant. The surfactants may be used singly or as a mixture of two or more thereof. Specific examples of the surfactant include polyoxyethylene alkyl phenyl ether, polyoxyethylene alkyl ether, polyoxyethylene sorbitan alkyl ester, alkyl pyridinium salts, higher alcohol sulfate ester salts, and the like.

In addition to the protease inhibitor, the chelating agent and the surfactant, the specimen pretreatment liquid can contain components that can be generally used by those skilled in the art. Examples include components constituting a buffer solution for maintaining pH 5 to 9 that is pH suitable for the reaction. An organic acid may also be appropriately contained.

Pretreatment of a specimen may be conducted in a usual manner. For example, a specimen can be pretreated by being mixed with the specimen pretreatment liquid. For example, a cotton-tipped swab into which a specimen such as rhinorrhea has been absorbed may be immersed in the specimen pretreatment liquid, followed by sufficiently mixing the specimen pretreatment liquid with the specimen, thereby pretreating the specimen. In this specification, the specimen which has been treated with the pretreatment liquid refers, for convenience, to “a sample”.

The sample obtained by treating the specimen with the specimen pretreatment liquid can be measured for SARS virus by an antigen-antibody reaction in a conventional immunoassay method. As used herein, SARS virus is a virus belonging to the SARS virus. The SARS virus also includes mutated SARS viruses.

The immunoassay method is a method of measuring a substance quantitatively by using the ability of an antibody to bind to the substance. Examples of the immunoassay method include radioimmunoassay (RIA), enzyme immunoassay (EIA), enzyme-linked immunosorbent assay (ELISA), fluorescence immunoassay (FIA), fluorescence polarization, and immunochromatography.

The antibody used in the immunoassay method may be an antibody that binds specifically to SARS virus. This antibody can be obtained using any conventional method. For example, the antibody can be obtained by the method for establishing hybridomas by cell fusion according to Kohler and Milstein (Kohler G and C. Milstein, Continuous cultures of fused cells secreting antibody of predefined specificity, Nature, 256: 495-497, 1975). The antibody may also be obtained by mere immunization of an animal with an antigen, followed by purification of its serum. The antibody used in the immunoassay method may be either a single antibody or a mixture of two or more antibodies.

The antibody includes fragments of the antibody, engineered antibodies thereof such as chimeric antibodies and humanized antibodies, and mutant antibodies thereof. These fragments, engineered antibodies or mutant antibodies also have the same specificity for SARS virus as that of the original antibody. These can also be produced by a means or method known to those skilled in the art.

Depending on the measurement method, the antibody may be labeled with a labeling substance or may be immobilized on a solid phase.

The labeling substance for labeling the antibody is suitably selected depending on the measurement method. For example, when the measurement method is radioimmunoassay (RIA), the labeling substance used is a radioisotope such as ¹²⁵I, ¹⁴C or ³²P. The labeling substance used in enzyme immunoassay (EIA) or enzyme-linked immunosorbent assay (ELISA) is an enzyme such as β-galactosidase, peroxidase, alkali phosphatase, or the like. The labeling substance used in fluorescent immunoassay (FIA) or fluorescence polarization is a fluorescent dye such as a fluorescein derivative or a rhodamine derivative. In immunochromatography, an insoluble granular marker or the like can be used.

The material or shape of a solid phase for immobilizing the antibody is appropriately selected depending on the measurement method. The material of the solid phase is not particularly limited insofar as it has high affinity for the antibody. Examples of the solid phase include synthetic organic polymer compounds such as polyvinyl chloride, polyvinylidene fluoride (PVDF), polystyrene, a styrene-divinyl benzene copolymer, a styrene-maleic anhydride copolymer, nylon, polyvinyl alcohol, polyacrylamide, polyacrylonitrile and polypropylene, polysaccharides such as a dextran derivative, agarose gel and cellulose, and inorganic polymer compounds such as glass, silica gel and silicone. These materials maybe those into which functional groups such as an amino group, an aminoacyl group, a carboxyl group, an acyl group, a hydroxyl group and a nitro group have been introduced. The shape of the solid phase is also appropriately selected depending on the measurement method. For example, the solid phase may be placoid (e.g., a microtiter plate (ELISA plate) and a disk), particulate (e.g., beads), tubular (e.g., a test tube and a tube), fibrous and membranous. The method of immobilizing the antibody on the solid phase may be a method known in the art, such as a physical adsorption method, an ionic bonding method, a covalent bonding method or an inclusion method.

As the immunoassay method, a highly sensitive measurement method using an enzyme, fluorescence or radioisotope is preferably used for detecting SARS virus contained in a very small amount in a specimen. Particularly, a safe and convenient enzyme immunoassay method is preferable. When the enzyme immunoassay method is used, a chemiluminescence substrate is preferably used as a substrate on which an enzyme acts. By measuring the chemiluminescence generated by the reaction of an enzyme with a chemiluminescence substrate, SARS virus can be measured accurately with high measurement sensitivity.

The specimen pretreatment liquid can be applied to a SARS measurement kit for measuring SARS virus in a specimen by an immunoassay method. In addition to the specimen pretreatment liquid, the SARS measurement kit contains first and second antibodies that bind specifically to SARS virus.

The SARS measurement kit is used to measure SARS virus in a specimen by the immunoassay method described above. The immunoassay method for the SARS measurement kit is preferably an enzyme immunoassay method. The immunoassay method is particularly preferably an enzyme immunoassay method using a chemiluminescence substrate.

The specimen pretreatment liquid in the SARS measurement kit contains the protease inhibitor described above. As the protease inhibitor, it is preferable to use at least one member selected from the group consisting of a serine protease inhibitor, acysteine protease inhibitor and an aspartic protease inhibitor.

The specimen pretreatment liquid in the SARS measurement kit can contain not only the protease inhibitor but also the above-mentioned chelating agent, surfactant and the like.

The specimen pretreatment liquid can be contained in one container as a liquid containing a protease inhibitor and other components. However, when the protease inhibitor is easily decomposable in an aqueous solution, a first solution containing a protease inhibitor dissolved in a water-soluble organic solvent and a second solution containing other components may be contained in separate containers, respectively. The storage stability of the protease inhibitor can thereby be improved. In this case, the first and second solutions maybe used by being mixed just before measurement. As the water-soluble organic solvent, it is possible to use ethanol, dimethylsulfoxide (DMSO), methanol, acetonitrile, 2-propanol or the like. Among these solvents, ethanol, dimethylsulfoxide (DMSO) and methanol are preferable.

At least one of the first and second antibodies contained in the SARS measurement kit is preferably an antibody that binds specifically to a SARS virus nucleocapsid protein (SARS-NP). This is because SARS-NP is relatively hardly mutated. Particularly the first and second antibodies are preferably those antibodies that bind specifically to different recognition sites of SARS-NP. This is because use of those antibodies binding specifically to different recognition sites of SARS-NP as the first and second antibodies leads to higher sensitivity and specificity and to improvement in detection accuracy.

The SARS virus measurement kit may further contain a solid phase for immobilizing the first antibody. This solid phase may be the same as the above-described solid phase. When the solid phase is contained in the SARS virus measurement kit, the solid phase may be contained therein with the first reagent containing a first antibody and the second reagent containing a second antibody. Alternatively, the solid phase (for example, a microplate) having a first antibody immobilized thereon can be contained in the kit.

In addition to those mentioned above, the SARS virus measurement kit may further contain, for example, a wash for washing wells of a microplate, a substrate for an enzyme labeling the second antibody, and the like. The wash includes a buffer solution containing a salt at a predetermined concentration.

The specimen pretreatment liquid of the present embodiment can be used in a SARS virus detection method for measuring SARS virus in a specimen by an immunoassay method. The method for detecting SARS virus comprises steps of: treating a specimen with an specimen pretreatment liquid containing a protease inhibitor to prepare a sample; forming a complex of SARS virus in the treated sample obtained by the treating step, a first antibody specifically binding to the SARS virus and a second antibody specifically binding to the SARS virus by binding the first antibody and the second antibody to the SARS virus; and detecting the complex obtained by the forming step.

In this method for detecting SARS virus, a specimen pretreatment liquid is used to treat a specimen in a specimen treatment step to obtain a sample. In the step of forming a complex, SARS virus in the sample is then reacted with the first and second antibodies binding specifically to SARS virus to form a complex consisting of the SARS virus as well as the first and second antibodies. That is, the method for detecting SARS virus is an immunoassay method with the measurement principle of the sandwich method.

When a solid phase for immobilizing the first antibody and a labeling substance for labeling the second antibody are used, the first antibody in the complex formed in the complex-forming step has been immobilized on a solid phase. The second antibody in the complex has been labeled with a labeling substance. Accordingly, the complex can be detected by detecting the labeling substance of the second antibody in the detection step.

In this detection method, the solid phase and the labeling substance described above in the immunoassay method can be used as the solid phase for immobilizing the first antibody and the labeling substance for labeling the second antibody, respectively. Examples of the solid phase include a microtiter plate, a plastic tube, and glass beads. Examples of the labeling substance include enzymes such as peroxidase, galactosidase, and alkali phosphatase. This labeling substance may be bound directly or indirectly to the second antibody. For example, the second antibody is labeled with biotin. Then, avidin that binds specifically to biotin is labeled with a labeling substance. Then, the second antibody can be labeled with the labeling substance via a biotin-avidin bonding.

Now, an immunoassay method using the detection method described above is described.

A capturing antibody (first antibody) against a measurement substance (SARS virus-derived antigen) is adsorbed onto a solid phase (microplate). Then, a sample obtained by treating a specimen with a pretreatment liquid is added to the microplate on which the first antibody has been immobilized. A complex is thereby formed between the measurement substance in the sample and the immobilized first antibody. Subsequently, a second antibody labeled with biotin is added. A complex consisting of the immobilized first antibody, the measurement substance and the biotin-labeled second antibody is thereby formed on the microplate. Thereafter, avidin labeled with a labeling substance (enzyme) is added to the microplate and bound to biotin of the second antibody. After washing, a substrate (chemiluminescence substrate) for the enzyme that is a labeling substance of avidin is added. Then, the chemiluminescence generated as a result of the enzyme reaction is measured to detect the measurement substance.

### EXAMPLES

The first antibody used in the Examples is a monoclonal antibody (referred to hereinafter as “first monoclonal antibody”) produced by a hybridoma that is mouse-mouse hybridoma SARS-23. Sysmex Corporation deposited this hybridoma with International Patent Organism Depositary (IPOD), National Institute of Advanced Industrial Science and Technology (AIST), ChuoDairoku, Higashi 1-1-1, Tsukuba City, Ibaraki Pref., Japan. This hybridoma was accepted on Feb. 15, 2005 (domestic accession date) under Accession No. FERM BP-10680. This hybridoma is a fusion cell between a mouse splenocyte and a mouse myeloma cell and produces an antibody (first monoclonal antibody) recognizing an epitope located in a region of positions 283 to 422 from the N-terminus of an amino acid sequence of SARS-NP.

The second antibody used in the Examples is a monoclonal antibody (referred to hereinafter as “second monoclonal antibody”) produced by a hybridoma that is mouse-mouse hybridoma SARS-12. Sysmex Corporation deposited this hybridoma with International Patent Organism Depositary (IPOD), National Institute of Advanced Industrial Science and Technology (AIST), Chuo Dairoku, Higashi 1-1-1, Tsukuba City, Ibaraki Pref., Japan. This hybridoma was accepted on Sep. 26, 2006, under Accession No. FERM BP-10687. This hybridoma is a fusion cell between a mouse splenocyte and a mouse myeloma cell and produces an antibody (second monoclonal antibody) recognizing an epitope located in a region of positions 1 to 141 from the N-terminus of an amino acid sequence of SARS-NP.

### Example 1

This example was conducted for the purpose of confirming the effect of a protease inhibitor in a specimen pretreatment liquid on the sensitivity of detection of SARS virus.

### (1) Preparation of a Specimen Pretreatment Liquid

20 μl of 0.1 M PMSF in ethanol was added to a solution (pH 7.0) containing 0.3 (v/v) % NP-40 (polyoxyethylene (9) octyl phenyl ether), 15 mM EDTA.2Na.2H₂O, 60 mM NaOH, 6 mM ACES (N-(2-acetamide)-2-aminoethanesulfonic acid), 0.22 M NaCl, and 15 mM NaN₃ (referred to hereinafter as solution A), to give an specimen pretreatment liquid 1.

10 μl of 1% inhibitor cocktail was added to the solution A, to give a specimen pretreatment liquid 2. This 1% inhibitor cocktail was prepared by dissolving Protease Inhibitor Cocktail (trade name) (Product No. P1860, Sigma-Aldrich) in purified water according to its protocol. The Protease Inhibitor Cocktail (trade name) contains a serine protease inhibitor aprotinin, a cysteine protease inhibitor E-64, a serine and serine protease inhibitor leupeptin, and an aspartic protease inhibitor pepstatin A.

### (2) Specimen and its Pretreatment

A mixture of roughly equal amounts of rhinorrheas collected from 5 healthy subjects was used as a specimen.

This specimen was mixed with the specimen pretreatment liquid 1, and the resulting mixture was filtered in an extraction bottle equipped with a filter for a commercial human influenza test kit, Poctem Influenza A/B (Sysmex Corporation). The resulting filtrate was mixed with His-tag-added recombinant SARS-NP (referred to hereinafter as recombinant SARS-NP) prepared by a method described in PCT/JP2006/320330, to give solutions containing recombinant SARS-NP at concentrations of 0, 3.125, 6.25, 12.5, 25, 50 and 100 pg/ml respectively. The resulting solutions were used as sample 1.

Separately, the specimen was mixed with the specimen pretreatment liquid 2, and the resulting mixture was filtered in the extraction bottle. The resulting filtrate was mixed with the recombinant SARS-NP to give solutions containing recombinant SARS-NP at concentrations of 0, 3.125, 6.25, 12.5, 25, 50 and 100 pg/ml respectively. The resulting solutions were used as sample 2.

Separately, the specimen was mixed with the solution A, and the resulting mixture was filtered in the extraction bottle. The resulting filtrate was mixed with the recombinant SARS-NP to give solutions containing recombinant SARS-NP at concentrations of 0, 3.125, 6.25, 12.5, 25, 50 and 100 pg/ml respectively. The resulting solutions were used as comparative sample 1.

Separately, the solution A was mixed, without using the rhinorrhea specimen, with the recombinant SARS-NP to give solutions containing recombinant SARS-NP at concentrations of 0, 3.125, 6.25, 12.5, 25, 50 and 100 pg/ml respectively. The resulting solutions were used as comparative sample 2.

### (3) Measurement Method

First, the first monoclonal antibody was immobilized onto a luminescence measurement ELISA plate in the following manner.

0.1 ml of a sensitization buffer prepared by diluting the first monoclonal antibody to a final concentration (immobilization concentration) of 1 μg/ml with 0.1 M phosphate buffer (pH 7.5) containing 0.1% sodium azide was added to each well of a luminescence measurement ELISA plate (manufactured by Nunc International). The plate was left overnight at 4° C. and then washed 3 times with 10 mM phosphate buffer containing 150 mM NaCl and 0.05% Tween 20 (hereinafter referred to as a plate wash), and 0.3 ml of 10 mM phosphate buffer (pH 7.0) containing 2.5 mM EDTA, 1% BSA, 150 mM NaCl and 5% casein (hereinafter referred to a blocking solution) was added to each well, and the plate was left overnight at 4° C.

50 μl of 10 mM phosphate buffer (pH 7.0) containing 2 mM EDTA, 1% BSA, 150 mM NaCl and 0.5% casein (hereinafter referred to as a sample diluent) was added to each well of the luminescence measurement ELISA plate on which the first monoclonal antibody had been immobilized. Then, 50 μl of each sample was added to each well and stirred for 1 hour at room temperature. The plate was washed 3 times with the plate wash, and then 100 μl of a labeled antibody solution prepared by diluting the biotin-labeled second monoclonal antibody to 1 μg /ml with the sample diluent was added to each well and reacted at room temperature for 30 minutes under stirring. Then, the plate was washed 3 times with the plate wash, and 100 μl of a peroxidase (POD)-labeled streptavidin solution prepared by diluting POD-labeled streptavidin to 0.02 μg/ml with the sample diluent was added to each well of the plate and stirred at room temperature for 30 minutes. The plate was washed 3 times with the plate wash, then a luminescence substrate (FEMTOGLOW (Funakoshi Co., Ltd.)) was added to each well and stirred, and the generated luminescence was measured with a luminescence plate reader, FLUOstarOPTIMA (BMG, Moritex Co., Ltd.).

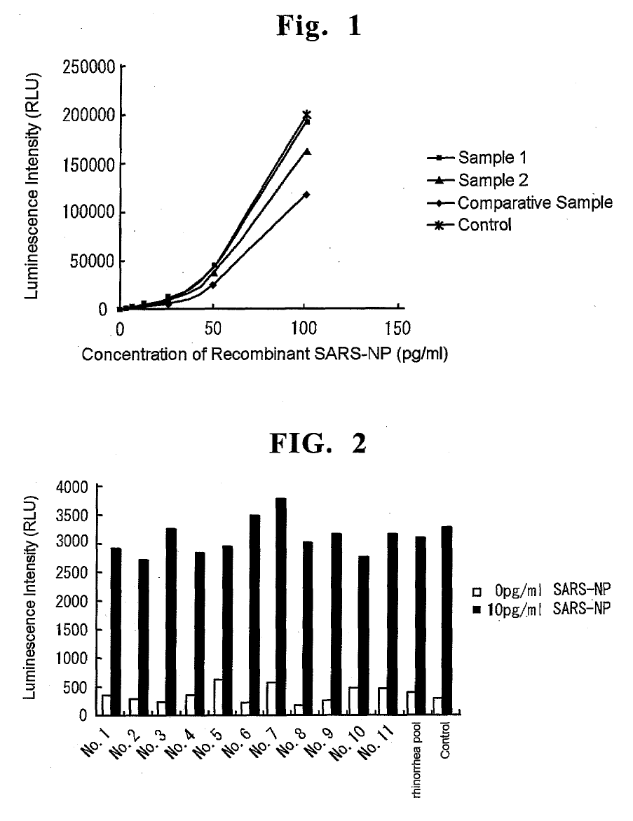

In FIG. 1, the comparative sample 1 showed lower luminescence intensity than that of the comparative sample 2. It was thereby revealed that a component contained in the rhinorrhea specimen exerts influence on the sensitivity of detection of SARS-NP. The samples 1 and 2 showed higher luminescence intensity than that of the comparative sample 1. Particularly, the sample 1 obtained by using the specimen pretreatment liquid 1 containing PMSF as a protease inhibitor showed luminescence intensity similar to that of the comparative sample 2. It was thereby inferred that the component that exerted influence on the sensitivity of detection of SARS-NP was a protease inhibitor contained in the rhinorrhea specimen. It was then found that by treating the rhinorrhea specimen with the pretreatment liquid containing a protease inhibitor, SARS virus could be measured with higher sensitivity by inhibiting the influence of a protease on SARS-NP.

### Example 2

This example was conducted for the purpose of examining whether the effect of the protease inhibitor varied due to the individual difference among rhinorrhea specimens.

Rhinorrheas (Nos. 1 to 11) collected from 11 healthy subjects, and the rhinorrhea mixture used in Example 1, were used as specimens.

Rhinorrheas Nos. 1 to 11 were treated with the specimen pretreatment liquid 1 and used to prepare a sample free of the recombinant SARS-NP (0 pg/ml) and samples (samples 1 to 11) containing the recombinant SARS-NP at 10 pg/ml in the same manner as in Example 1, and the luminescence intensity of each sample was measured.

Separately, the rhinorrhea mixture was treated with the specimen pretreatment liquid 1 and used to prepare a sample free of the recombinant SARS-NP (0 pg/ml) and a sample (sample 12) containing the recombinant SARS-NP at 10 pg/ml in the same manner as in Example 1, and the luminescence intensity of each sample was measured.

Samples (comparative samples) were prepared in the same manner as described above except that the solution A was used in place of the specimen pretreatment liquid 1, and their luminescence intensities were similarly measured. The results are shown in FIG. 2.

In FIG. 2, the variation in measurements of the samples 1 to 11, attributable to the individual difference among the rhinorrhea specimens in the samples, was low and all of these 11 samples were recognized to be subject to the same effect of the protease inhibitor as on the sample 12 and the comparative sample. It was thus found that the effect of the protease inhibitor contained in the pretreatment liquid could be obtained regardless of the individual difference among the rhinorrhea specimens.

### Example 3

This example was conducted for the purpose of examining whether SARS virus in rhinorrhea specimens collected from SARS patients could be detected by the detection method using the pretreatment liquid.

Rhinorrheas collected from 18 patients with SARS, and rhinorrheas from 20 healthy subjects, were used as specimens.

Each specimen was mixed with the specimen pretreatment liquid 1, and the resulting mixture was filtered in the extraction bottle described in Example 1. The resulting filtrate was used as a sample and measured for its luminescence intensity by the same measurement method as in Example 1 except that the concentration of the POD-labeled streptavidin solution was 0.1 μg/ml.

For comparison, the amount of SARS virus RNA in the specimen was also measured by the RT-PCR method. Extraction of RNA from the specimen and synthesis of cDNA for PCR were conducted by a method described in Poon LL, et al. Early diagnosis of SARS Coronavirus infection by real time RT-PCR, J Clin Virol 2003; 28:233-8. PCR with synthesized cDNA was conducted according to a method described in Poon LL, et al. Detection of SARS Coronavirus in Patients with Severe Acute Respiratory Syndrome by Conventional and Real-Time Quantitative Reverse Transcription-PCR Assays, Clin Chem. Januayr 2004;50(1): 67-72.

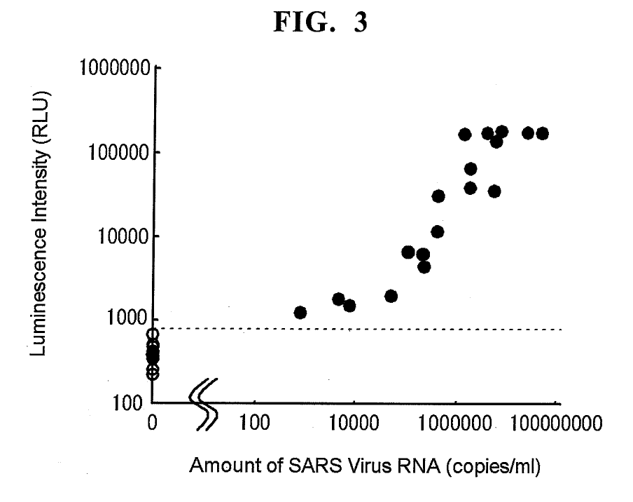

The results are shown in FIG. 3. In FIG. 3, the luminescence intensity obtained by the immunoassay method using the pretreatment liquid is shown on the ordinate. The amount of SARS virus RNA obtained by the RT-PCR is shown on the abscissa. The broken line in FIG. 3 shows a cutoff value (718 RLU).

FIG. 3 shows that when the cutoff value (718 RLU) indicated by the broken line in the graph is established for luminescence intensity, the luminescence intensity of every sample prepared from rhinorrheas of the healthy subjects is lower than the cutoff value, while the luminescence intensity of every sample prepared from rhinorrheas of the SARS patients is higher than the cutoff value. In all the samples prepared from rhinorrheas of the SARS patients, there was a correlation between the amount of SARS virus RNA obtained by the RT-PCR method and the luminescence intensity obtained by the immunoassay method using the pretreatment liquid. From the foregoing, it was found that SARS virus could be detected accurately by the detection method using the specimen pretreatment liquid.

The foregoing detailed description and examples have been provided by way of explanation and illustration, and are not intended to limit the scope of the appended claims. Many variations in the presently preferred embodiments will be obvious to one of ordinary skill in the art, and remain within the scope of the appended claims and their equivalents.

What is claimed is:

1. A pretreatment liquid for preparing a sample for measuring a virus included in rhinorrhea or sputum by an immunoassay method using an antibody specifically binding to the virus, comprising a protease inhibitor for inhibiting an influence of a protease to the virus.

2. The pretreatment liquid according claim 1, wherein the virus is SARS virus.

3. The pretreatment liquid according claim 1, wherein the immunoassay method is an enzyme immunoassay method.

4. The pretreatment liquid according to claim 3, wherein the enzyme immunoassay method uses chemiluminescence substrate.

5. The pretreatment liquid according to claim 1, wherein the protease inhibitor is at least one selected from serine protease inhibitor, cysteine protease inhibitor and aspartic protease inhibitor.

6. The pretreatment liquid according to claim 5, wherein the serine protease inhibitor is phenyl methyl sulfonyl fluoride.

7. The pretreatment liquid according to claim 1 further comprising a chelating agent.

8. The pretreatment liquid according to claim 1 further comprising a surfactant.

9. The pretreatment liquid according to claim 1, wherein the antibody specifically bind to a nucleocapsid protein of the virus.

10. A kit for measuring a virus included in rhinorrhea or sputum by an immunoassay method, comprising:
a pretreatment liquid comprising a protease inhibitor for inhibiting an influence of a protease to the virus;
a first antibody specifically binding to the virus; and
a second antibody specifically binding to the virus.

11. The kit according to claim 10, wherein the immunoassay method is an enzyme immunoassay method.

12. The kit according to claim 11, wherein the enzyme immunoassay method uses a chemiluminescence substrate.

13. The kit according to claim 10, wherein the protease inhibitor is at least one member selected from a serine protease inhibitor, a cysteine protease inhibitor and an aspartic protease inhibitor.

14. The kit according to claim 10, wherein the first and second antibodies are specifically bind to a different recognition site of a nucleocapsid protein of the virus respectively.

15. The kit according to claim 10 further comprising a solid phase for immobilizing the first antibody.

16. The kit according to claim 10, wherein the first antibody is included in a first reagent and the second antibody is included in a second reagent.

17. The kit according to claim 15, wherein the first antibody is immobilized to the solid phase.

18. A method for detecting a virus included in a specimen including rhinorrhea or sputum, comprising steps of:
treating the specimen with a pretreatment liquid comprising a protease inhibitor;
forming a complex of the virus in the treated specimen obtained by the treating step, a first antibody specifically binding to the virus and a second antibody specifically binding to the virus by binding the first antibody and the second antibody to the virus; and
detecting the complex obtained by the forming step.

19. The method according to claim 18, wherein the first antibody of the complex is immobilized to a solid phase.

20. The method according to claim 18, wherein the second antibody is labeled by labeling substance, and the detecting step is performed so as to detect the complex by detecting the labeling substance of the second antibody included in the complex.
